(19) United States
(12) Patent Application Publication
(10) Pub. No.: US 2015/0353467 A1
(43) Pub. Date: Dec. 10, 2015

(54) 3,4-DIAMINOPYRIDINE TARTRATE AND PHOSPHATE, PHARMACEUTICAL COMPOSITIONS AND USES THEREOF

(71) Applicant: ASSISTANCE PUBLIQUE-HOPITAUX DE PARIS, Paris (FR)

(72) Inventors: Francois GUYON, Paris (FR); Dominique PRADEAU, Nogent-sur-Marne (FR); My Dung LE HOANG, Paris (FR); Jean-Jacques HOURI, Le Perreux sur Marne (FR)

(21) Appl. No.: 14/818,848

(22) Filed: Aug. 5, 2015

Related U.S. Application Data
(63) Continuation of application No. 14/085,017, filed on Nov. 20, 2013, now abandoned.
Application No. 14/085,017 is a continuation of application No. 10/467,082, filed on Jan. 20, 2004, now abandoned.

(51) Int. Cl. C07C 59/255; A61K 31/44; A61K 45/06; C07D 213/73

(52) U.S. Cl. CPC C07C 59/255; C07D 213/73; A61K 31/44; A61K 45/06

(57) ABSTRACT

The invention relates to 3,4-diaminopyridine salts, pharmaceutical compositions containing at least one of said salts and uses thereof for the treatment of botulism, myasthenia, myasthenic syndromes or fatigue.

The present invention relates to salts of 3,4-diaminopyridine, to pharmaceutical compositions comprising at least one of its salts, and to their uses.

The use of pyridine derivatives in the pharmaceutical field has been widely described for various applications and in particular for the treatment of myasthenia or myasthenic syndromes.

The fact common to myasthenia and to the various myasthenic syndromes currently known is exertion-induced fatigability. It is possible to distinguish, on the one hand, myasthenia (myasthenia gravis: MG), which is an autoimmune disease, and, on the other hand, myasthenic syndromes, which are an assemblage of highly disparate conditions:
Lambert-Eaton myasthenic syndrome (LEMS), which can be regarded as of autoimmune origin but which is not necessarily a paraneoplastic syndrome,
congenital myasthenia,
myasthenic syndromes of medicinal or toxic origin.

All these conditions result from attack on the neuromuscular junction at different levels.

For example, it has been described that certain pyridine derivatives, such as 4-aminopyridine (4-AP) and 3,4-diaminopyridine (3,4-DAP), can be used for the treatment of myasthenia and myasthenic syndromes because they improve neuromuscular transmission by increasing the entry of cellular calcium, which promotes the release of acetylcholine in the nerve endings (Murray N. M. et al., Neurology, 1981, 31, 265-271).

The paper by McEvoy K. M. et al. (N. Engl. J. Med., 1989, 321, 1567-1571) indicates, in this respect, that 3,4-DAP can be effectively used for the treatment of Lambert-Eaton myasthenic syndrome.

Furthermore, animal studies have suggested that, in this specific application, 3,4-DAP would be more effective and would have fewer side effects than 4-AP (Lemeighan M. et al., Brain Res., 1984, 304, 166-169, and Paskov D. S. et al., Eksp. Khir. Anestexiol., 1973, 18, 48-52).

Furthermore, the ability of 3,4-DAP to increase the release of acetylcholine in the nerve endings also makes it possible to envisage its use in improving the cognitive functions during aging (U.S. Pat. No. 4,386,095).

Provision has also already been made to use 3,4-DAP for the symptomatic treatment of fatigue related to a neurological pathology, such as, for example, multiple sclerosis (Bever et al., Annals of Neurology, 1990, 27, 421-427, and Sheean et al., Brain, 1998, 121, 967-975).

Finally, the use of aminopyridines, and in particular of 3,4-DAP, has already been provided, in particular in U.S. Pat. No. 5,952,357, for the treatment of diseases affecting motor neuron cells, such as acute infectious poliomyelitis and its effects, Creutzfeldt-Jakob syndrome, some toxic and nutritional disorders, such as those related to vitamin B12 deficiency, degeneration of motor neurons as a result of exposure to certain compounds, such as aluminum, or degenerative diseases, such as amyotrophic lateral sclerosis, primary lateral sclerosis, presenile dementia with attack on motor neurons, spinal muscular atrophies, olivoponto-cerebellar atrophy, Joseph's disease, Parkinson's disease, Huntington's chorea or Pick's disease.

However, although the therapeutic effectiveness of 3,4-DAP has been recognized, the use of this compound, in the free base form, in medicinal forms results in very short periods of validity of the corresponding medicament, stored under the stability conditions in accordance with the international recommendations (International Conference on Harmonization, Draft Guideline on Stability Testing of New Drug and Product, November 1999, CPMP/ICH/2736/99). Thus, it is not currently possible to envisage the marketing of 3,4-DAP.

It is in order to overcome these problems that, surprisingly, the Inventors have developed that which forms the subject matter of the invention.

The Inventors therefore set themselves the target of supplying novel compounds having therapeutic properties at least equivalent to those of 3,4-DAP in the free base form but exhibiting an improved stability over time, in particular after they have been incorporated in a medicinal form, and have discovered that certain salts of 3,4-DAP allow this target to be met.

A subject matter of the present invention is thus specific salts of 3,4-diaminopyridine, characterized in that they are chosen from 3,4-diaminopyridine tartrate and phosphate.

This is because the Inventors have shown that 3,4-DAP tartrate and phosphate exhibit a very high stability under the storage conditions in accordance with the international recommendations, whereas, under these same conditions, 3,4-DAP is unstable.

These specific salts of 3,4-DAP also exhibit greater solubility in water, thus facilitating their formulation.

These compounds are provided in the form of a white crystalline powder which is very soluble in water.

These compounds can be prepared according to a preparation process which consists in reacting 3,4-DAP with tartaric acid or phosphoric acid, in order to obtain the corresponding 3,4-DAP tartrate or phosphate.

Another subject matter of the invention is a pharmaceutical composition including, as active principle, 3,4-diaminopyridine tartrate or phosphate and optionally at least one pharmaceutically acceptable vehicle.

The pharmaceutical composition in accordance with the invention exhibits the properties of being able to be used in the same indications as 3,4-DAP, such as, for example, for the treatment of botulism, myasthenia, myasthenic syndromes and fatigue related to a neurological pathology, such as, for example, multiple sclerosis or amyotrophic lateral sclerosis.

The pharmaceutical composition in accordance with the invention can be administered by the oral route, taken 3 to 4 times daily in chronic use, or by the injectable route.

The pharmaceutical composition in accordance with the invention can therefore be provided in various forms, such as in the form of hard gelatin capsules, of capsules, of compressed tablets, of suspensions to be taken orally, of lozenges or of injectable solutions or in any other form appropriate to the method of administration by the oral or injectable route.

The amount of 3,4-DAP tartrate or phosphate present in the pharmaceutical composition in accordance with the invention preferably corresponds to unit doses of between 5 mg and 20 mg, expressed as weight of 3,4-DAP in the free base form.

The nature of the pharmaceutically acceptable vehicle optionally present in the pharmaceutical composition in accordance with the invention will, of course, vary according to the method of administration and the pharmaceutical presentation of said composition.

The pharmaceutical vehicle is generally composed of one or more excipients conventionally used for the preparation of pharmaceutical compositions, such as antiagglomerating agents, antioxidants, dyes, vitamins, inorganic salts, taste-modifying agents, smoothing agents, coating agents, isolating agents, their mixtures and generally any excipient conventionally used in the pharmaceutical industry.

Of course, a person skilled in the art will take care on this occasion that the additive or additives optionally used are compatible with the intrinsic properties attached to the pharmaceutical composition in accordance with the invention.

The pharmaceutical composition in accordance with the invention can furthermore include one or more additional active principles.

Finally, a subject matter of the invention is the use of 3,4-diaminopyridine tartrate or phosphate in the preparation of a pharmaceutical composition intended for the treatment of botulism, myasthenia, myasthenic syndromes or fatigue related to a neurological pathology, such as multiple sclerosis or amyotrophic lateral sclerosis.

In addition to the preceding provisions, the invention also comprises other provisions which will emerge from the description which will follow, which refers to two examples of the preparation of 3,4-DAP tartrate and phosphate and to an example relating to the study of the stability of 3,4-DAP tartrate.

However, it should be clearly understood that these examples are given solely by way of illustration of the subject matter of the invention, of which they do not in any way constitute a limitation.

### EXAMPLE 1

### Preparation of 3,4-diaminopyridine tartrate

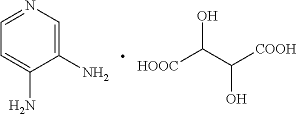 [SMILES: Nc1ccncc1N ; O=C(O)C(O)C(O)C(=O)O]

100 parts of 3,4-DAP (Aldrich), purified beforehand, and 300 parts of distilled water are introduced into a reactor. The mixture is brought to the boiling point with stirring.

Separately, 135.5 parts of L-tartaric acid are dissolved in 200 parts of distilled water. The solution of L-tartaric acid is slowly introduced into the refluxing solution of 3,4-DAP. The reaction mixture is subsequently left at reflux for 15 minutes and then it is cooled to 70° C.

1 part of active charcoal powder is then added and stirring is continued for 15 minutes. The charcoal is subsequently separated by filtration.

The colorless solution is subsequently cooled gradually to 40° C. and held at this temperature for 12 hours with stirring. After draining and washing with 50 parts of absolute ethanol, the product is dried at 60° C. under vacuum to constant weight. 164 part of 3,4-DAP tartrate are then obtained, the melting point of which is between 178 and 180° C. The 3,4-DAP tartrate can subsequently be repurified by crystallization from water.

The elemental analysis of the product thus obtained was carried out on a Perkin-Elmer CHN 4000 device. The product sample is weighed on a balance with an accuracy of 10⁻⁴ mg; the percentage of oxygen was calculated by difference.

The elemental analysis of the product obtained, in accordance with that of the expected product, is as follows:

| % | C | H | N | O |
|---|---|---|---|---|
| Calculated | 41.70 | 5.06 | 16.21 | 37.03 |
| Found | 41.71 | 5.07 | 16.22 | 37.00(*) |

(*)calculated by difference: [100 − (C % + H % + N %]

### EXAMPLE 2

### Preparation of 3,4-diaminopyridine phosphate

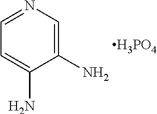

### Stage 1): synthesis of 3,4-DAP phosphate

90 parts of 3,4-DAP (Aldrich), purified beforehand, and 1 800 parts of distilled water are introduced into a reactor. The mixture is brought to a temperature of 75° C. with stirring. Dissolution is observed to be complete.

Subsequently, 191 parts of 85% phosphoric acid are slowly introduced into the 3,4-DAP solution. After the addition of the phosphoric acid, the reaction mixture is kept at a temperature of 80° C. for a further 15 minutes and is then cooled to 35° C.

The reaction mixture is then kept at a temperature of between 30 and 35° C. for 4 hours with stirring.

The precipitate formed is drained and washed with 100 parts of distilled water and then with 100 parts of absolute ethanol. After drying under vacuum at 60° C. to constant weight, 160 parts of crude 3,4-DAP phosphate are obtained in the form of a white powder, the melting point of which is between 225 and 227° C.

### Stage 2): purification of the crude 3,4-DAP phosphate

160 parts of crude 3,4-DAP phosphate obtained above in stage (1), 640 parts of absolute ethanol and 715 parts of distilled water are introduced into a reactor. The mixture is heated, with stirring, to a temperature of 80° C. At this temperature, dissolution is complete.

The reaction mixture is subsequently cooled gradually to a temperature of 4° C. and is held at this temperature for 12 hours with stirring.

After draining and washing with 100 parts of absolute ethanol, 180 parts of wet product are obtained. The product is subsequently dried at 60° C. under vacuum to constant weight. 133 parts of 3,4-DAP phosphate are then obtained, the melting point of which is 229° C.

The elemental analysis of the product obtained was carried out under the same conditions as those described above in example 1.

The elemental analysis of the product obtained, in accordance with that of the expected product, is as follows:

| % | C | H | N | P | O |
|---|---|---|---|---|---|
| Calculated | 28.99 | 4.83 | 20.29 | 14.97 | 30.92 |
| Found | 29.05 | 4.93 | 20.23 | Not | Not |
| | | | | determined | determined |

### EXAMPLE 3

### Stability study on 3,4-diaminopyridine tartrate

3,4-DAP tartrate, as prepared above in example 1, was introduced into hard gelatin capsules made of gelatin of size No. 3 in a proportion of 10 mg (expressed as weight of 3,4-DAP in the free base form) per hard gelatin capsule (Hard gelatin capsules A).

In the same way and by way of comparison, hard gelatin capsules made of gelatin of size No. 3 including 10 mg of 3,4-DAP per hard gelatin capsule were prepared (Hard gelatin capsules B).

A stability study on 3,4-DAP tartrate and on 3,4-DAP, introduced respectively into hard gelatin capsules A and B, was subsequently carried out.

This study was carried out under the conditions recommended by the international recommendations for the study of the stability of active principles (International Conference on Harmonization, Draft Guideline on Stability Testing of New Drug and Product, November 1999, CPMP/ICH/2736/99).

The results obtained appear in tables I and II below:

TABLE I

| | | | T = 3 months (storage at | |
|---|---|---|---|---|
| | T = 0 | | 25° C., relative humidity of 60) | |
| | Appearance | Content of | Appearance | Content of |
| | of the | active | of the | active |
| | powder | principle (*) | powder | principle |
| Capsule A | crystalline | 95.6 | crystalline | 97.1 |
| (3,4-DAP | white | | white | |
| tartrate) | | | | |
| Capsule B | crystalline | 98.5 | yellow | not |
| (3,4-DAP) | white | | (decompo- | determined |
| | | | sition product) | |

(*) percentage of the target value

TABLE II

| Storage conditions: | | | |
|---|---|---|---|
| Temperature: 40° C. | | Capsule A (3,4- | Capsule B (3,4- |
| Relative humidity: 75% | | DAP tartrate) | DAP) |
| T = 0 | Appearance of the | Crystalline | Crystalline |
| | powder | white | white |
| | Content of active | 95.6 | 98.5 |
| | principle (*) | | |
| T = 3 | Appearance of the | Crystalline | Yellow |
| months | powder | white | (decomposition |
| | | | product) |
| | Content of active | 96.1 | Not determined |
| | principle (*) | | |
| T = 6 | Appearance of the | Cream-colored | Yellow |
| months | powder | powder | (decomposition |
| | | | product) |
| | Content of active | 97.9 | Not determined |
| | principle (*) | | |

(*) percentage of the target value

These results show that 3,4-DAP tartrate, formulated in hard gelatin capsules, exhibits great stability, whereas 3,4-DAP is unstable.

We claim:

1. A stable salt of 3,4-diaminopyridine, selected from the group consisting of from 3,4-diaminopyridine tartrate and 3,4-diaminopyridine phosphate, wherein the salt is stable at 6 months when stored at 40 C and 75% relative humidity.

2. A pharmaceutical composition, comprising, a stable salt of 3,4-diaminopyridine tartrate or phosphate, wherein the salt is stable at 6 months when stored at 40 C and 75% relative humidity and at least one pharmaceutically acceptable vehicle.

3. The pharmaceutical composition as claimed in claim 2, in the form of hard gelatin capsules, of capsules, of compressed tablets, of oral suspensions, of lozenges or of injectable solutions.

4. The composition as claimed in claim 2 wherein the amount of 3,4-DAP tartrate or phosphate present corresponds to unit doses of between 5 mg and 20 mg, expressed as weight of 3,4-DAP in the free base form.

5. The composition as claimed in claim 2, comprising a pharmaceutically acceptable vehicle selected from the group consisting of antiagglomerating agents, antioxidants, dyes, vitamins, inorganic salts, taste-modifying agents, smoothing agents, coating agents, isolating agents and their mixtures.

6. The composition as claimed in claim 2, further comprising one or more additional active principles.

7. A method of treating a condition, comprising administering to a patient in need thereof, an effective amount of the pharmaceutical composition of claim 2, wherein said condition is one or more selected from the group consisting of botulism, myasthenia, myasthenic syndromes and fatigue related to a neurological pathology.

8. The method of claim 7, wherein the condition is multiple sclerosis or amyotrophic lateral sclerosis.

9. The composition as claimed in claim 3, wherein the amount of 3,4-DAP tartrate or phosphate present corresponds to unit doses of between 5 mg and 20 mg, expressed as weight of 3,4-DAP in the free base form.

10. The composition as claimed in claim 3, comprising a pharmaceutically acceptable vehicle selected from the group consisting of antiagglomerating agents, antioxidants, dyes, vitamins, inorganic salts, taste-modifying agents, smoothing agents, coating agents, isolating agents and their mixtures.

11. The composition as claimed in claim 4, comprising a pharmaceutically acceptable vehicle selected from the group consisting of antiagglomerating agents, antioxidants, dyes, vitamins, inorganic salts, taste-modifying agents, smoothing agents, coating agents, isolating agents and their mixtures.

12. The composition as claimed in claim 3, further comprising one or more additional active principles.

13. The composition as claimed in claim 4, further comprising one or more additional active principles.

14. The composition as claimed in claim 5, further comprising one or more additional active principles.

15. A method of treating Lambert-Eaton myasthenic syndrome, comprising orally administering a tablet pharmaceutical composition comprising a stable 3,4 diaminopyridine phosphate salt in an amount that is equivalent to 5 mg to 20 mg of 3,4 diaminopyridine, and a pharmaceutically acceptable vehicle.
